(19) United States
(12) Patent Application Publication
(10) Pub. No.: US 2017/0007446 A1
(43) Pub. Date: Jan. 12, 2017

(54) METHOD AND DEVICE FOR MEASURING THE POSITION OF AN EYE

(71) Applicant: Carl Zeiss Meditec AG, Jena (DE)

(72) Inventors: Michael Stefan Rill, Jena (DE); Delbert Peter Andrews, Oberkochen (DE)

(21) Appl. No.: 15/113,756

(22) PCT Filed: Jan. 26, 2015
(86) PCT No.: PCT/EP2015/051431

§ 371 Date: Jul. 22, 2016

(30) Foreign Application Priority Data
Jan. 31, 2014 (DE) 102014201746.7

(51) Int. Cl. A61F 9/008; A61B 3/12; A61B 3/113; A61B 3/00; A61B 3/10

(52) U.S. Cl. CPC A61F 9/008; A61B 3/0025; A61B 3/102; A61B 3/113; A61B 3/1225; A61F 2009/00851; A61F 2009/00846; A61B 2090/3735

(57) ABSTRACT

A device for measuring the position of an eye of a mammal. To determine a positional change of the eye between two time points, the device comprises: at least one optical coherence tomograph for generating images of at least a part of the retina at the two time points and for emitting corresponding image data and an image processing device which is designed to compare the image data assigned to the two time points and to determine an angle of rotation between the images, wherein the image processing device is additionally designed to output the angle of rotation as information about a cyclotorsion of the eye between the two time points.

### PRIORITY CLAIM

The present application is a National Phase entry of PCT Application No. PCT/EP2015/051431, filed Jan. 26, 2015, which claims priority from German Patent Application Number 102014201746.7, filed Jan. 31, 2014, the disclosures of which are hereby incorporated by reference herein in their entirety.

### FIELD OF THE INVENTION

The invention relates to a device as well as a method for measuring the position of an eye of a mammal, wherein a change in position between two time points is determined.

### BACKGROUND OF THE INVENTION

In ophthalmology, various surgical interventions are known for improving or restoring vision for the application of which the exact position of the eye must be known. Examples are the refractive correction of defective vision by means of modification of the cornea or methods for the modification of an intraocular lens. A further example is the implantation of an intraocular lens to correct astigmatism, i.e. a toric intraocular lens. As visual defects are usually not rotationally symmetrical, but often also comprise an astigmatism, the principal meridians of the eye, i.e. the cutting planes of the steepest and flattest curvature, need to be known as precisely as possible for surgery. At least one principal meridian is therefore determined diagnostically before the surgical intervention. At that time, the patient usually sits on a chair in an upright position. During the surgical intervention, the patient is in a supine position. It is known that, in particular when a person lies down, the eye rotates about the axis of the fovea. In scientific literature, this rotation is called cyclotorsion, and the angle of rotation is different from patient to patient and also depends on the change in position.

It is known in the state of the art to mark a reference axis to which the diagnostically determined principal meridians relate on the cornea, for example by means of a marker or a cutting device. This marking is performed in such a way that the mark is visible during the surgical intervention and serves as an alignment aid.

Marking the eye is not only time-consuming and prone to errors, there is also the risk of a marking being smudged by tear fluid or by a saline solution used during the operation with the result that the position of the reference axis can no longer be determined exactly. Cut markings do not have this problem but are difficult to recognize with conventional operating microscopes.

It is known as a further development in the state of the art to detect the cyclotorsion of the eye by analyzing images of the iris. In this regard, reference is made to the following publications: U.S. 2009/0012505 A1; S. Arba-Mosquera, M. Arbelaez, “Three-month clinical outcomes with static and dynamic cyclotorsion correction using the Schwind Amaris”, Cornea, September 2011, 30 (9), p. 951-957; S. Arba-Mosquera, M. Arbelaez, “Use of a six-dimensional eye-tracker in corneal laser refractive surgery with the Schwind Amaris TotalTech laser”, J. Refract. Surg., August 2011, 27 (8), p. 582-590. This state of the art images the iris of the eye repeatedly, recognizes structures in the iris and uses these to determine the cyclotorsion of the eye. The pupil center is determined and used as center of rotation of the cyclotorsion.

Such measurement proves to be problematic when the patient's pupil is dilated by medication. Pattern recognition of the iris structure then becomes difficult to impossible.

### SUMMARY OF THE INVENTION

Therefore, it is an object of the invention to provide a method and a device for measuring the position of an eye of a mammal in the form of determining a change in position between two time points which makes it possible to give information about a cyclotorsion of the eye which can be determined without problems before an operation and also works without errors in the case of pupils dilated by medication.

The object is achieved according to the invention by a device for measuring the position of an eye of a mammal which, to determine a change in position of the eye between two time points, comprises: at least one optical coherence tomograph for generating images of at least a part of the retina at the two time points and for outputting image data assigned to the two time points, and an image processing device which is adapted to compare the image data assigned to the two time points and to determine an angle of rotation between the images, wherein the image processing device is further adapted to output the angle of rotation as information about a cyclotorsion of the eye between the two time points.

The object is further achieved by a method for measuring the position of an eye of a mammal, wherein a change in position of the eye between a first time point and a second time point is determined in that, by means of optical coherence tomography, a first image of at least a part of the retina is obtained at the first time point and a second image of the part of the retina is obtained at the second time point, an angle of rotation between the first and second image is determined and the angle of rotation is outputted as information about cyclotorsion of the eye occurred between the first and second time point.

According to the invention, optical coherence tomography is used in order to image at least a part of the retina at least twice, namely during the diagnosis of the eye, i.e. the determination of the principal meridians in the case of determining astigmatism, and directly before the surgical intervention. From the imaging of the part of the retina, image rotation is determined which provides information about the cyclotorsion of the eye. The assessment of the retina is possible without problems even in the case of a dilated pupil because the reduction in the size of the iris caused by the dilation of the pupil does is irrelevant to measurement of cyclotorsion. Furthermore, the procedure according to the invention does not require any physical marking of the patient's eye by means of a cutting device or a marker and thus avoids the disadvantages associated therewith. A further advantage is a time saving for the attending doctor since, as a rule, modern diagnostic devices image the eye to be treated anyway by means of optical coherence tomography. The repetition of this imaging before the surgical intervention is therefore a simple means for also using the image data obtained during the diagnosis at the same time to determine the cyclotorsion.

The type of optical coherence tomograph is not decisive for the principle according to the invention. SS-OCT, FD-OCT and TD-OCT come equally into consideration. If FD-OCT is used it is possible to accelerate the method by dispensing with the Fourier transform normally. The inventors have recognized that the rotational position can already be determined by a corresponding data comparison on the basis of the untransformed raw data of the OCT. Within the meaning of the invention, the term “image data” is therefore understood to also comprise raw data which do not yet provide any suitable image but are original data on which observable images are generated. In particular, the term “image data” also comprises raw data from reflection and/or scattered light measurements and the raw data of an FD-OCT before the Fourier transform. The use of raw data makes it possible to scan the retina more quickly.

In a preferred embodiment, the optical coherence tomograph is designed to carry out a retina scan although it is sufficient to image only a part of the retina. The cyclotorsion of the eye is a rotation about the fovea. It is therefore particularly preferred that the imaged part of the retina comprises the fovea. Blood vessels extend from this fovea into the choroid membrane of the eye. If the position of the fovea and the position of these blood vessels is determined, the angle of rotation can be determined particularly easily if the fovea is taken as center of rotation and the positions of the extending blood vessels is determined at the two time points. In this way, the angle of rotation can be established by means of a simple image comparison.

The position of the blood vessels in the choroid membrane of the eye can be determined particularly preferably by an edge detection since in this way the structures can be recognized particularly easily.

In eye surgery, a surgical microscope is usually used which has a display and a control device. It is preferred to design it in such a way that it superimposes the angle of rotation on the display. It is particularly preferred to relate this angle of rotation to a reference axis which in turn refers to the astigmatism of the eye. The reference axis can, for example, be the axis of a principal meridian.

In laser-assisted eye surgery, laser treatment devices are commonly used which emit laser radiation to target points lying in the eye. Examples are the so-called LASIK operation with ablation of the cornea by emission of laser radiation onto a plurality of different target points, the generation of laser-assisted incisions in or close to the limbus to correct astigmatism (limbal relaxing incisions) or the generation of a cut surface in the eye through laser radiation emitted to target points. For such laser treatment devices, embodiments of the invention are advantageous which generate control data. These control data are, of course, in the case of an optical correction which takes into account an astigmatism, based on the rotational position of the eye, i.e. generally based on the principal meridians. It is preferred to correct the control data on the basis of the information about the cyclotorsion of the eye which has occurred between the two time points.

The information about the cyclotorsion of the eye can be determined continuously during a surgical intervention on an eye in order to achieve tracking with respect to a change in the information about cyclotorsion of the eye. In this way, eye movements can be compensated for.

Insofar as device features are referred to in this description, these features also apply of course analogously to the corresponding method. Equally, method features described here also correspond to functional features of the device described here. The device can realize corresponding functional features in particular with respect to the image processing device and/or any control devices via programming.

It is understood that the features named above and those yet to be explained below can be used not only in the stated combinations but also in other combinations or alone, without departing from the scope of the present invention.

### BRIEF DESCRIPTION OF THE DRAWINGS

The invention is explained in even more detail below by way of example with reference to the attached drawings, which also disclose features essential to the invention. There are shown in:

FIG. 1 a schematic representation of a diagnostic device for measuring a patient's eye before a refractive correction of astigmatism,

FIG. 2 a schematic representation of a treatment device for the refractive correction of astigmatism, and

FIG. 3 two images which are obtained and assessed in measurements of the cyclotorsion of the eye.

### DETAILED DESCRIPTION

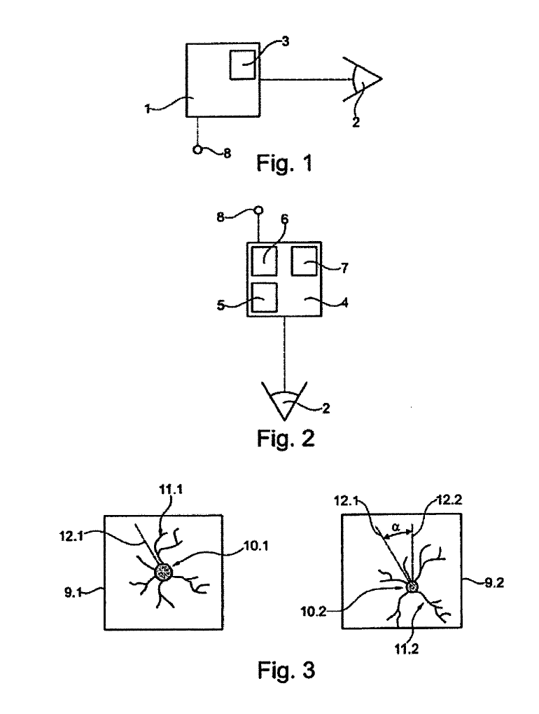

FIG. 1 shows schematically a diagnostic device 1 for the diagnostic examination of an eye before surgery to correct defective vision in which the embodiment example described involves a LASIK operation. The diagnostic device 1 senses an eye 2 the defective vision of which is to be corrected. For this, the diagnostic device 1 contains an optical coherence tomograph, OCT 3 for short, which comprises an axial sensing range from the cornea to the retina of the eye 2. In other words, the OCT 3 is in a position, depending on the setting, not only to survey the cornea of the eye 2 but also to obtain an image of the retina of the eye 2.

The need for correction of the eye 2 is determined by the diagnostic device 1. An astigmatism is also detected which, as is usual in ophthalmology, is indicated with respect to the position of the principal meridian (position of the steepest meridian). Alternatively, information based on the flattest meridian can be used. The patient sits in front of the diagnostic device 1, i.e. is in an upright position. With the OCT 3, not only is the position of the principal meridian determined but also an image of the retina of the eye 2 is obtained and stored. The corresponding measurement values or data which were determined by the diagnostic device 1 can be made available to other devices, for example an associated surgical microscope, via a data connection 8. This is explained further below.

FIG. 2 shows schematically a treatment device 4 which can optionally also be provided as surgical microscope 4. This device equally comprises an optical coherence tomograph in the form of OCT 5. The patient lies under the treatment device 4/operating microscope 4. Because of this change in position, cyclotorsion occurs in the eye, i.e. the eye rotates about the optical axis. In order to determine this rotation, the OCT 5 records an image of the retina of the eye. A control device 6 compares this image with the image which was provided by the diagnostic device 1. This image can, for example, be imported via the data connection 8 mentioned. The angle of rotation which the eye has performed in cyclotorsion about the optical axis can be easily determined from the image comparison.

For this, in a first embodiment, the treatment device 4 is provided with a control device 6 which, on the one hand, carries out the image analysis mentioned and, on the other hand, activates a laser treatment device 7 which changes structures in the eye within the framework of an ophthalmic intervention. If, on the other hand, the device is designed as surgical microscope 4, the laser treatment device 7 does not need to be part of the device and the control device 6 can also be formed as a simple image processing device. In a modification of the construction of devices 1 and 4, these can also be combined in one unit. Then, an external data connection in the form of the data connection 8 is not necessary and only a single OCT is used.

FIG. 3 shows two images corresponding to image data 9.1 and 9.2 which are provided by OCT 3, 5. In the embodiment represented of FIGS. 1 and 2, OCT 3 provides the image data 9.1 and OCT 5 provides the image data 9.2. As already explained, the image data are captured at different time points, namely the image data 9.1 during the diagnostic examination of the eye and the image data 9.2 directly before the surgical intervention on the eye. In the embodiment without separated devices 1 and 4, both the image data 9.1 and the image data 9.2 originate from the same OCT, but likewise at different time points. The nerve head 10.1, 10.2 of the retina of the eye 2 can be recognized in the image data 9.1, 9.2. The optical axis of the eye 2 runs through the fovea or at least almost through the fovea. It therefore represents a good approximation of the center of rotation in cyclotorsion. The fovea 10.1 and 10.2 is therefore taken as center of rotation as a basis in the described image analysis. The angle of rotation is determined from a detection of blood vessels 11.1, 11.2 of the choroid membrane of the eye 2. It is preferably referenced to a principal axis 12.1 of an astigmatism which was determined during the diagnostic examination, i.e. at the time point of obtaining the image data 9.1. The angle of rotation α of the cyclotorsion results in this principal axis being rotated by the angle α at the second time point, i.e. at the capture of the image data 9.2, wherein the center of rotation is the fovea 10.1, 10.2.

By means of an edge detection, the control device 6 (or the image analysis device in the case of the realization of the device as surgical microscope 4) determines the angle of rotation α and makes this available for subsequent processes. The angle of rotation α can be made available for the correction of target data of the laser treatment device 7 (device formed as treatment device 4) or another laser treatment device (device formed as operating microscope 4). The image data 9.1 and 9.2 show not only a rotation but also a lateral displacement. This is of no further relevance for the determination of the angle of rotation α, i.e. the information about the cyclotorsion, since the fovea 10.1, 10.2 is adopted as center of rotation. Usually, the information about the cyclotorsion therefore comprises not only the angle of rotation α but also the position of the center of rotation, i.e. the point at which the optical axis passes through the retina.

The information about the cyclotorsion can be determined once before the start of the surgical intervention. This information can be used to correct control data for the laser treatment device 7 or another laser treatment device which were generated from the information obtained a time point image data 9.1 were acquired. In a further development, the device is also active during the surgical intervention in that the information about the cyclotorsion is acquired continuously and is used to update control of the laser treatment device with respect to varying cyclotorsion.

The angle of rotation can be determined, for example, by means of image registration. A possible embodiment of such an image registration is the use of a correlation function. For this, an image area around the nerve head is selected and the correlation function is formed for different relative rotational positions of this area of the image data 9.1 and 9.2. A maximum correlation function value is obtained for the negative angle of rotation α, i.e. when the image data 9.2 are rotated backwards by exactly the value of a into the position of the image data 9.1.

The information about the cyclotorsion, for example the angle of rotation, the center of rotation and preferably also about the change of the principal axis 12.1 to the principal axis 12.2 is preferably superimposed on or suitably overlaid on a display of the surgical microscope 4 or of the treatment device 5.

1. A device for measuring the position of an eye of a mammal which, for the determination of a change in position of the eye between two time points, comprises:
at least one optical coherence tomograph for generating images of at least a part of the retina at the two time points and for outputting image data assigned to the two time points and
an image processing device is adapted to compare the image data assigned to the two time points and to determine an angle of rotation between the images, wherein the image processing device is further adapted to output the angle of rotation as information about cyclotorsion of the eye that occurred between the two time points.

2. The device according to claim 1, wherein the at least one optical coherence tomograph is adapted to carry out a retina scan.

3. The device according to one of the above claim 1, wherein the at least one optical coherence tomograph is adapted in such a way that the imaged part of the retina comprises a nerve head, and the image processing device is adapted to determine the image data, the position of the nerve head and the position of blood vessels extending from the fovea in the choroid membrane of the eye and takes a fovea as center of rotation for determining the angle of rotation (α).

4. The device according to claim 3, wherein the image processing device is adapted to determine, in the image data, the position of the blood vessels in the choroid membrane of the eye by means of an edge detection.

5. The device according to claim 1, which further comprising a surgical microscope having a display and a control device which superimposes the angle of rotation on the display based on a reference axis of an astigmatism of the eye.

6. The device according to claim 1, further comprising a control device for generating control data for a laser treatment device or in which the image processing device is provided as a control device for generating control data, wherein the laser treatment device emits laser radiation to target points lying in the eye in order to effect changes in structures of the eye, wherein the control device corrects the control data on the basis of the information about the cyclotorsion of the eye.

7. The device according to claim 1, further comprising a laser treatment device which emits laser radiation to target points lying in the eye in order to effect changes in structures of the eye, wherein the optical coherence tomograph repeatedly generates the image data and the image processing device repeatedly outputs the information about the cyclotorsion of the eye and the laser treatment device tracks the target points with respect to a change in the information about the cyclotorsion of the eye.

8. A method for measuring a position of an eye of a mammal, wherein a change in position of the eye between a first time point and a second time point is determined, the method comprising
using an optical coherence tomography to obtain a first image of at least a part of a retina at the first time point and to obtain a second image of the part of the retina at the second time point,
determining an angle of rotation between the first and second image and
outputting the angle of rotation as information about cyclotorsion of the eye that occurred between the first and second time point.

9. The method according to claim 8, wherein of the optical coherence tomography is used to carry out a retina scan.

10. The method according to claim 8, wherein the part of the retina comprises a fovea of the retina, and wherein a position of the fovea and a position of blood vessels in the choroid membrane of the eye are determined and the nerve head is taken as centre center of rotation as a basis for determining the angle of rotation.

11. The method according to claim 10, wherein the position of the blood vessels in the choroid membrane of the eye is determined by edge detection.

12. The method according to claim 11, wherein the angle of rotation is superimposed on a display of a surgical microscope, preferably based on a reference axis of an astigmatism of the eye.

13. The method according to claim 8, wherein further control data are generated for a laser treatment device which emits laser radiation to target points lying in the eye in order to effect changes in structures of the eye, and wherein the control data are corrected on the basis of the information about the cyclotorsion of the eye.

14. The method according to claim 8, wherein the position of the mammal is repositioned between the first and second time point such that the orientation of the optical axis of the eye is changed perpendicular to the horizontal about an angle of 60-120°.
